(19) United States
(12) Patent Application Publication
(10) Pub. No.: US 2026/0047977 A1
(43) Pub. Date: Feb. 19, 2026

(54) DEVICE AND METHOD OF USE FOR MEASURING AND MODIFYING THE ORBITAL AND INTRAORBITAL CONTENT, VOLUME, PULSALITY, LYMPHATIC DRAINAGE, VENOUS DRAINAGE, AND BLOOD FLOW

(71) Applicant: Tarek A. Shazly, Pittsburgh, PA (US)

(72) Inventor: Tarek A. Shazly, Pittsburgh, PA (US)

(21) Appl. No.: 19/100,441

(22) PCT Filed: Sep. 12, 2023
(86) PCT No.: PCT/US2023/032553

§ 371 Date: Jan. 31, 2025

Related U.S. Application Data
(60) Provisional application No. 63/406,211, filed on Sep. 13, 2022.

(51) Int. Cl. A61H 5/00; A61F 9/02; A61H 9/00

(52) U.S. Cl. CPC A61H 5/00; A61F 9/029; A61H 9/0071; A61H 2201/165; A61H 2201/5007; A61H 2201/5071; A61H 2230/045

(57) ABSTRACT

A device and method of use for measuring and modifying orbital content volume, pressure, blood flow, venous drainage, lymphatic drainage, and pulsatility of a subject's eye socket(s) (orbits) are provided. The device includes goggles including at least one chamber in front of the orbit that serves to expand the functional orbital volume to include the orbital contents and the space inside the chamber, at least one pressure measuring sensor, means for controlling the pressure inside the chamber such as a release valve, pump and/or vacuum device in fluid communication with the chamber, a processing and control mechanism, means of communication, and a power supply. The control mechanism can be operatively coupled to the pump, vacuum, release valve device and can maintain a predetermined pressure or pressure modulation in response to the measured pressure in the chamber when the device is worn by a subject.

### CROSS-REFERENCE TO RELATED APPLICATION(S)

This application claims priority to, and the benefit of, co-pending U.S. Provisional Application 63/406,211, filed Sep. 13, 2022, for all subject matter common to both applications. The disclosure of said provisional application is hereby incorporated by reference in its entirety.

### FIELD OF THE INVENTION

The present invention relates generally to ocular, extraocular, orbital, and cranial pressure, blood flow, lymphatic drainage, muscle contractility diagnostics, and treatments. In particular, and by non-limiting example, the present invention relates to the provision of a device that measures and modifies an orbital pressure and intraorbital content pressure of a patient's one or both orbits, correlating the measurements, and/or synchronizing the pressure with patient parameters, such as cardiac cycle.

### BACKGROUND

The term orbit refers to the cavity or socket of the skull in which the eye and its appendages are situated. It can refer to the bony socket or it can also be used to imply its contents. In the adult human, the average volume of the orbit is about 30 milliliters (1.01 US fl oz), of which the eye occupies 6.5 ml (0.22 US fl oz). The orbital contents comprise the eyeball, the orbital connective tissue and fascia, extraocular muscles, cranial nerves II (the optic nerve with its surrounding meningeal sheath and cranial cerebrospinal fluid (CSF) cuff), III (oculomotor), IV (trochlear), V (trigeminal), and VI (abducent), blood vessels, fat, the lacrimal gland with its sac and nasolacrimal duct, the eyelids, and medial and lateral eyelid ligaments. The orbit is connected to the cranial cavity (the inside of the skull) via openings called the superior orbital fissure, the inferior orbital fissure, and the optic canal allowing the nerves, vessels, and CSF around the nerve to connect to the cranial cavity.

Some of the orbital contents do change in volume either following the heart beats (cardiac cycle) such as orbital and ophthalmic arteries, veins, and CSF around the optic nerve. It is estimated that the eyeball volume goes up and down by around 280 microliters with each heartbeat. Other structures are contractile, such as the lid closing muscle (orbicularis oculi), and the extraocular muscles, such as the eyelid opening muscle (levator muscle), recti, and oblique extraocular muscles, which can change their volume during spontaneous, reflex, and voluntary contractions, such as blinking and eye movements. Additionally, the compartment of CSF surrounding the optic nerve enclosed in its meningeal covering transmits the CSF pulsations of the cranial cavity. The intracranial CSF pulsatility can also be transmitted into the orbit indirectly via controlling venous drainage from the orbit and determining intraorbital blood volume. In some pathological conditions where there is abnormal communication between the contents of the cranium and the orbit, such as encephalocele, the CSF pulsations in the orbit are more evident.

The orbit serves as a compartment with rigid bony walls except for the front end, which has soft tissues mostly formed by the eyelids, the eyeball, and its covering skin (the conjunctiva). Conditions that increase the pressure inside the orbit cause these structures to protrude forward due to the rigidity of the orbital walls.

Measurement of orbital pulse pressure is currently only available with an invasive technique involving a manometer connected to a needle that needs to be advanced into orbital soft tissues. Measurement of the change in orbital volume (waveform) can be used to determine the contractility of the orbicularis muscle, eyelid elevator muscle, and other extraocular muscles. Additionally, it can be used to measure the transmitted CSF pulsations transmitted to the orbit from the intracranial cavity along the optic nerve. Furthermore, the rhythmic pulsatility of the orbit following the cardiac cycle can indicate ocular and orbital blood flow through the internal carotid artery. Further diagnostic information can be gathered by comparing the readings from both orbits.

Several CSF disorders, including hydrocephalus, may be accompanied by changes in the pulsatile intracranial pressure (ICP). Altered pressure pulsatility provides information about intracranial pressure-volume reserve capacity, usually referred to as intracranial compliance (ICC). Impaired ICC may be associated with neuronal dysfunction and causes the intracranial compartment to be more responsive to intracranial volume changes. The temporary intracranial volume load provided by every heartbeat therefore results in increased pulsatile ICP, which can be diagnosed by ICP monitoring with single ICP wave analysis since the veins of the orbit, such as the superior and inferior ophthalmic veins, traverse the bony orbital apex and are bathed in the CSF. The ICP pulsatility is transmitted into the orbital contents via the ophthalmic veins. Furthermore, a compartment of CSF is enclosed in the sheath around the optic nerve. Transmitted ICP pulsations through the continuous CSF space can be measured indirectly via measuring the change in the volume of the intraorbital contents.

The intraocular pressure (IOP) of the eye is determined by the balance between the amount of aqueous humor (a transparent water-like fluid containing low protein concentrations secreted from the ciliary body that supports the lens of the eyeball) produced by the eye and the ease with which it leaves the eye primarily through the canal of Schlemm to the episcleral collector channels and episcleral veins. The Goldmann equation that predicts IOP states: IOP in millimeters of mercury (mmHg)=(rate of aqueous formation/facility of outflow)+episcleral venous pressure.

The aqueous humor drained by the episcleral collector channels follows two routes: the lymphatic drainage route into ocular lymphatic channels, and the venous drainage route into the superior ophthalmic vein.

Schlemm's canal is considered to be either a lymphatic vessel or a vascular channel as it exhibits structural and functional properties of both the vascular and lymphatic systems. It has been proven by Johannes Grüntzig et al. using different radioactive tracers that the aqueous humor of the anterior chamber of the eyeball and subconjunctival space drains predominated over the superficial cervical lymph nodes. In further investigations by means of interstitial dye lymphography, intraluminal projections were found similar to a zipper. These are believed to control the aqueous humor flow into a one-way flow. That directional flow is explained by the flow model developed by Gerhart Liebau. Conjunctival lymphatics show intraluminal structures by double contrast injection. They should play an important role in the regulation of the intraocular pressure, or the balance between production and outflow of the aqueous humor.

Historically, the ophthalmic veins are frequently thought to be devoid of valves. However, in a study by John Zhang et al, twelve superior ophthalmic veins were examined by stereomicroscopy. Ten valves were identified in nine (75%) superior ophthalmic vein specimens: four valves were in the superior ophthalmic vein and the remainder were located near its origin from angular and supra-orbital tributaries. The orientation of valve cusps predicted the following blood flow in the superior ophthalmic vein, as they were found to be towards the cavernous sinus. The authors concluded the existence of valves in the superior ophthalmic vein and its two main tributaries. These findings suggest a one-way flow of blood carrying the aqueous humor in the direction of the cavernous sinus and then to the heart via the internal jugular vein. This valved venous system then contributes significantly to the facility/ease of drainage of the aqueous humor. Furthermore, the superior ophthalmic vein is the main venous drainage channel of the orbital contents. Facilitation of the emptying of the superior ophthalmic vein into the intracranial cavernous sinus can aid the perfusion/blood flow to the intraorbital contents such as the eyeball as the blood flow to the tissues improve in response to lowering the venous pressure. As the perfusion pressure is the difference between the inflow Pi and outflow pressure Po, measured at the organ level.

When an intermittent positive external pressure is applied to the ocular lymphatics as well as the superior ophthalmic vein and its tributaries, the one-way valve structures in them will aid a uni-directional flow to help empty these drainage vessels. This results in aiding the venous drainage and lymphatic drainage of the orbital contents, helps reduce the intraocular pressure, and improves the orbital blood flow. It is ideal for the applied external pressure to be synchronized with the naturally occurring positive intraluminal pressure associated with arterial pulsatility to augment the pulsatile flow through these drainage vessels.

Poor blood flow to the eye is implicated in a number of ophthalmic and systemic disorders such as carotid artery disease, central retinal artery occlusion, central retinal vein occlusion, ocular ischemic syndrome, glaucoma, non-arteritic anterior ischemic optic neuropathy, and arteritic anterior ischemic optic neuropathy, as well as systemic disorders such as diabetes causing diabetic retinopathy and systemic hypertension causing hypertensive retinopathy.

Glaucoma is a common blinding disorder caused by elevated pressure inside the eye leading to impairment of blood flow to the optic nerve as well as mechanical damage to the optic nerve fibers due to pressure gradient between the front part of the nerve subjected to intraocular pressure and the back of the nerve bathed in CSF subjected to intracranial pressure (translaminar pressure). Ocular pulse amplitude (OPA) is the difference between systolic and diastolic intraocular pressure. OPA is known to vary between normal subjects and patients with glaucoma. OPA might help facilitate the drainage of the fluid inside the eye (aqueous humor) through the drain at the angle of the eye (trabecular meshwork).

Similar damage to the optic nerve can happen in conditions causing elevated intracranial pressure such as pseudotumor cerebri or idiopathic intracranial hypertension which also causes an increase in the translaminar pressure. The current standard of care for the measurement of intracranial pressure remains to insert a needle in the CSF space in the lower spine in a technique called lumbar puncture which is an invasive technique.

A magnitude of pathological conditions affecting the eyeball and other intraorbital contents results from impairment of blood flow, abnormal translaminar pressure across the optic nerve head, and poor drainage of aqueous humor due to poor ocular pulse amplitude.

### SUMMARY

There is a need for a device and corresponding method of use to address issues with blood flow impairment, abnormal translaminar pressure across the optic nerve, and poor aqueous humor drainage. The present invention is directed toward further solutions to address these needs, in addition to having other desirable characteristics.

A potential therapeutic approach to these conditions is to apply external pressure, whether positive or negative, to the orbital contents to achieve different therapeutic effects. Furthermore, modulating the orbital pressure to oppose or accentuate its natural pulsatility can also be utilized to modify factors, such as intraocular pressure, aqueous humor drainage, and orbital/ocular blood flow.

The present invention provides a device and method of use for measuring orbital volume change over time as indicated by the change in the pressure inside a chamber formed over the orbit by goggles. Additionally, the present invention modifies the orbital vascular and lymphatic channel content volume via changing the functional orbital volume (volume of orbital contents plus the volume of the chamber in front of the orbit formed by the goggle) modifying directly and indirectly the orbital and intraorbital contents pressure and parameters such as ocular pulse amplitude in a patient's subject's one or both orbits. Furthermore, the present invention enables adjusting the functional orbital volume and/or applying positive or negative pressure synchronized or asynchronized with the pre-existing orbital pulsatility profile and/or some patient vital measurements, such as cardiac cycle, without the need for invasive diagnostic devices, therapies, or use of medications.

The concept of ocular plethysmography has been disclosed in an article by E. C. Brockenbrough, et al., Review of Surgery July-August 1967, pages 299-302, which is addressed to the measurement of relative volume change in an ocular globe at constant vacuum as an indication of blood flow in the ophthalmic artery.

William Gee (U.S. Pat. No. 3,911,903A) discloses a device and method to enable the physician to make a determination of the intra-arterial pressure at which each eye begins to pulse at a specifically determinable level of vacuum via a suction cup attached to the white (sclera) of the eyeball.

Orbital plethysmography enables the measurement of changes in the orbital pulsatility at a specifically determinable level of pressure and enables the determination of the amplitude of pulse volume in each orbit as related to a volumetric change in the orbital contents. Thus, when applied as a diagnostic tool to the orbit, it is possible to indirectly determine the adequacy of internal carotid artery flow as a reflection of the degree of stenosis in the respective carotid artery.

The technique known as tonometry determines the intraocular pressure. The technique known as ophthalmodynamometry enables the determination of the systolic and diastolic blood pressure within the central retinal artery by applying an external force to the eye while directly visualizing the back of the eye for visible pulsatility of the retinal vessels. Ophthalmodynamometry enables the determination of the orbital pulse volume change and the simultaneous determination of the orbital blood pressure at which these pulse volume changes occur via applying high enough pressure to the orbit to stop blood flow similar to sphygmomanometer (blood pressure cuff). The technique known as ocular plethysmography enables the determination of pulsatile volume changes of the ocular globe.

Extraocular muscles include six muscles attached to the eyeball to control eye position, four straight muscles call the recti muscles and two oblique muscles called the obliques. Additionally, there is a muscle called the levator palpebrae superioris, which contracts to raise the upper eyelid and to maintain the upper eyelid position. On the other hand, orbicularis oculi is the muscle encircling the eyelids and is responsible for voluntary and reflex eyelid closure such as blinking. Certain pathological conditions can cause impairment of the contractility of these muscles. For example, third, fourth, and sixth cranial nerve palsies can cause weakness of eye movements in specific directions. Third cranial nerve palsy can additionally cause eyelid drooping and seventh nerve palsy can cause the inability to close the eyelid. Other disorders such as myasthenia gravis can cause autoimmune weakness of these muscles and cause variable exam findings. Evaluation and documentation of the contractility of these muscles are critical for the diagnosis and follow-up of patients with these disorders. Before now, there were only subjective and semi-objective ways of documenting these findings. For example, seventh nerve palsy (Bell's Palsy) causing weakness of the eyelid closure (orbicularis occuli) is currently diagnosed subjectively by an eye doctor or a neurologist using a subjective scale of no lid closure, weak, moderate, good closure. Another example is the evaluation of eyelid muscle fatigability to diagnose myasthenia. A physician asks the patient to open and close the eyelids repeatedly looking for a subjective decline in the eyelid muscle strength over time. Alternatively, an invasive test can be used (single fiber electromyogram) to stimulate a muscle fiber around the eyelids with repetitive electric stimulus and measure a gradual decline in the muscle fiber recorded electric activity without being able to measure the actual contraction. In comparison, weak hand muscles can be easily and objectively examined and recorded using simple non-invasive methods. It can be quantified by measuring the amount of static force that the hand can squeeze around a dynamometer. The force has most commonly been measured in kilograms and pounds, but also in milliliters of mercury and in Newtons. Similar means are required to record the contractility (strength) of the extraocular muscles including the levator and orbiculari oculi.

In the technique referred to as opthalmodynamography reference to the ocular orbit is intended to include the bony socket in which resides the ocular globe or eyeball, the extraocular muscles, the periorbital tissues, and the eyelids with its contained tissues.

The present invention provides an apparatus and corresponding method of use for the determination of volume changes in a portion of the human body for example, the orbit(s) indirectly indicating the extent and nature of blood flow, ocular volume change, and muscle contractility.

In addition, the present invention provides an apparatus for the relative measurement of orbital pressure indicated by volume changes in the orbit along with the cardiac cycle while the orbit is subjected to normal atmospheric pressure, sub-atmospheric pressure, or above atmospheric pressure without cessation of blood flow.

In addition, the present invention provides an apparatus for the modification of the functional orbital volume (total volume of the orbital contents in addition to the space contained by the goggles in front of the orbit) via changing the chamber volume to modify the orbital content volume and their pressure and/or by applying a dynamic positive or negative change in the volume of the functional orbital volume to attenuate or exaggerate the orbital pulsatility along with subject vitals such as the cardiac cycle. The change in functional orbital volume causes expansion and/or collapse of the compressible intraorbital contents such as blood vessels, lymphatic vessels, collector channels, and canal of Schlemm in response to increasing and decreasing the chamber volume respectively. Such modification of pulsatility can help improve blood flow, venous drainage, lymphatic drainage, or aqueous humor drainage through the trabecular meshwork to improve the blood circulation to the intraorbital structures and lower intraocular pressure.

In addition, the present invention provides an apparatus or device for detection of orbital pulsatility and its change with heart rate, posture, and breathing cycle. This information can be used to diagnose vascular, central nervous system, and pulmonary disorders.

In addition, the present invention provides an apparatus or device for comparing the blood flow through the two internal carotid arteries and detecting delay in vascular filling comparing the two orbits.

Furthermore, the present invention provides an apparatus for measuring orbital pulse waveform which indirectly indicates the intracranial pressure pulsatility which shapes the orbital pulsatility via transmitted CSF pulsations along the optic nerve and controlling the venous drainage from the orbit.

The present invention additionally provides an apparatus for measurement of the orbital pressure changes induced by voluntary, spontaneous, reflex, or triggered ocular, extraocular, and lid movements while the orbit is subjected to normal atmospheric pressure and comparison of the symmetry of these contractions by induced volume changes between the two orbits.

In accordance with embodiments of the present invention, a device or apparatus is provided for determining the change in orbital volume as an indication of a change in orbital content volume, such as a resultant blood flow to a particular part of a human body. The device or apparatus includes a cavity external to the orbit that freely communicates with one or more chambers attachable to the orbit, in which there are provided one or more pressure transducer means for detecting pressure or volume changes in the external cavity system in response to changes in the volume or pressure of the orbit. A volume control means is provided for adjusting the volume of the external chamber system. A means to trigger extraocular movements a venting means to reset the pressure system to atmospheric pressure on demand, a means to detect vital signal, a means to process the different signals and control the venting means and vacuum/pump means, and a recording means for continuously recording the pressure changes caused by the volume changes are also provided.

In accordance with an example embodiment of the invention, the pressure changes recorded are those pressure changes due to spontaneous pulsatile change in the orbital content volume with breathing and heartbeats, voluntary or reflex changes in the orbital volume due to change in posture, vascular occlusion, and muscle contraction under investigation.

In accordance with an example embodiment of the invention, the pressure changes recorded are those pressure changes due to spontaneous, reflex, voluntary, or induced muscle contraction in the orbital content.

In accordance with an example embodiment of the invention, the volume change of the orbital contents causing pressure changes in the chamber in front of the orbit(s) is indirectly measured using means to measure the pressure such as differential pressure transducer(s).

In accordance with an example embodiment of the invention for determining the relative blood flow in the two internal carotid arteries, the apparatus includes means to simultaneously record orbital volume changes as indicated by pressure changes in the chamber enclosed by the goggle cups attached to each orbit comparing the readings of the two orbits.

The device or apparatus and corresponding method of operation or use in accordance with the present invention include variations of eyecup, eyecups, goggle or goggles, configured to fit over one or both of a patient's orbits with means to measure the pressure inside the cup or goggle chamber and means to alter a pressure inside one or more cavities of the cup or goggle. An outer rigid surface of the goggle or goggles can seal against a patient's skin around a perimeter of his/her eye sockets using a belt, elastic band, or adhere to it using adhesive tape. The goggle cup is rigid apart from a window of flexible material or an outlet to a secondary chamber that has some flexible walls allowing the change in the volume via mechanical means. Alternatively, the cup is held against the peri-orbital skin using variable force so it can be held tighter or looser resulting in smaller functional orbital volume versus larger orbital volume respectively. Subsequently, when the means to alter functional orbital volume is actuated, a pressure differential from atmospheric pressure can be created and maintained inside the one or more goggle cavities and over one or both orbits. The cavity pressure can be either increased or decreased relative to atmospheric pressure via changing the chamber volume, depending on the desired therapeutic effect (e.g., whether glaucoma is being treated or whether papilledema is being treated). The cavity pressure can be modulated in response to the prerecorded measurements of the change in orbital pressure whether synchronized or asynchronized with the prerecorded waveform. Additionally, the modulation of the functional orbital volume can be determined by other measured vital data of the subject such as pulse oximetry, electric activity of the heart obtained via electrocardiogram (EKG), head position, breathing cycle, blinking, and/or eye movement tracking. Furthermore, the modulation can aim at exaggerating or diminishing the pre-treatment orbital pulsatility. Asynchronized modulation of the cavity volume can be utilized to improve the venous return from the orbit and diminish the episcleral venous pressure of the eyeball to aid in aqueous drainage and subsequently lowering IOP.

According to an example embodiment of a device, a goggle or goggles can be configured to fit over one or both orbits of a subject and seal against the peri-orbital skin, around the eye sockets. The goggle or goggles can be sufficiently airtight to allow the formation of a chamber contained behind the goggle cup that is in direct communication with the orbit and allows measuring the orbital content volume change as indicated by the change in the air pressure inside the goggle or goggles and modulating the intraorbital content volume via changing the functional orbital volume which in turn changes the air pressure inside the chamber in front of the orbits.

The mechanism to measure the pressure consists of at least one pressure transducer such as a piezoelectric pressure sensor to measure the pressure in the chamber. The mechanism to modulate the functional orbital volume, which in turn modulates the pressure inside the goggle chamber(s) can be a vacuum, pump, vacuum-pump, or means to change the volume of the chamber behind the goggles chamber(s). Such means can include but are not limited to a collapsible, flexible chamber connected to the fixed chamber with mechanical means to compress or expand the collapsible chamber, and/or means to hold the cup against the peri-orbital skin using variable force so it can be held tighter or looser compressing the soft accordion-like cushion lining the chamber rim against the periorbital skin resulting in smaller functional orbital volume versus larger orbital volume respectively. Such means may include linear actuators, solenoids, voice-coil actuators, and piezo electric actuators.

Additional mechanical means, fixation targets, other goggle or remote biosensors such as EKG sensors, pulse oximetry, gyroscopic sensors to sense the head position in space, as well as means to contract or record extraocular muscle movements such as electromyograms, gaze trackers, and Electronic Muscle Stimulation (EMS) respectively are additional parts of various embodiments of the current invention.

The power supply powering the system can be a rechargeable battery or other wired or wireless power source integral to the goggle or goggles. Control means allow displaying the measured parameters, transmitting data to an external unit for example a computer in wired or wireless communications, and means to control the functional orbital pressure modulation to achieve the desired therapeutic effect.

When an intermittent positive external pressure is applied to the ocular lymphatics as well as the superior ophthalmic vein and its tributaries, the one-way valve structures in them aid a uni-directional flow to help empty these drainage vessels. This results in aiding the venous drainage and lymphatic drainage of the orbital contents and helps reduce the intraocular pressure and improve the orbital blood flow. It is ideal for the applied external pressure to be synchronized with the naturally occurring positive intraluminal pressure associated with arterial pulsatility to augment the pulsatile flow through these drainage vessels.

The device can go through two phases, the measurement phase where it measures the orbital pressure curve, and correlates it with other simultaneously measured vitals, such as EKG or pulse oximetry to time it against the cardiac cycle, once such data is registered via the device's processing unit; and the treatment phase where either positive, negative or alternating positive and negative pressure(s) is applied to modulate the orbital pressure in sync or out of sync with the cardiac cycle measured via the biometric sensors, such as pulse oximeter or EKG. Attenuated or delayed arterial flow waves in the measurement phase can be augmented or attenuated by applying negative or positive pressure at the time of the treatment phase to achieve the intended therapeutic effect(s).

These and other examples and features of the device of the present invention and its use will be set forth in part in the following Detailed Description. This Overview is intended to provide non-limiting examples of the present subject matter—it is not intended to provide an exclusive or exhaustive explanation. The Detailed Description below is included to provide further information about the present assemblies and methods.

### BRIEF DESCRIPTION OF THE FIGURES

These and other characteristics of the present invention will be more fully understood by reference to the following detailed description in conjunction with the attached drawings, in which:

FIG. 1 is a cross-sectional view of an orbit.

FIG. 2 is a side schematic view of a device positioned in front of an orbit in accordance with embodiments of the present invention;

FIG. 3 is an isometric view of a device in accordance with embodiments of the present invention;

FIG. 4 is a view of a device in accordance with embodiments of the present invention;

FIG. 5 is a simplified lateral cross-sectional view of the skull showing the orbit and intracranial cavity in accordance with embodiments of the present invention;

FIG. 6 is a simplified lateral cross-sectional view of the orbit and intraorbital contents in accordance with embodiments of the present invention;

FIG. 7 is a simultaneous recording of orbital pressure tracing along with pulse oximetry tracing of a subject in accordance with embodiments of the present invention;

FIG. 8 is a simultaneous recording of orbital pressure tracing along with pulse oximetry tracing of a subject at baseline and following jugular vein occlusion in accordance with embodiments of the present invention;

FIG. 9 is a side view of the contents of the orbit with the device placed in front of the orbit in accordance with embodiments of the present invention;

FIG. 10 is a cross section of the superior ophthalmic vein and/or one of its tributaries. A, under atmospheric or subatmospheric pressure during the diastolic phase of the heart cycle and B, under atmospheric or subatmospheric pressure during the systolic phase of the heart cycle, and C when an external pressure is applied to the vein walls facilitating the flow of blood towards the heart in accordance with embodiments of the present invention; and

FIG. 11 is a flow chart showing the steps of measuring orbital pulsatility and applying treatment protocol for intended therapeutic effects in accordance with embodiments of the present invention.

### DETAILED DESCRIPTION

An illustrative embodiment of the present invention relates to a device and corresponding method of use to address issues with blood flow impairment, abnormal translaminar pressure across the optic nerve head, and poor aqueous humor drainage. The device can comprise variations of eyecup, eyecups, goggle or goggles, configured to fit over one or both of a patient's orbits with means to measure the pressure inside the cup or goggle chamber and means to measure and modify a pressure inside one or more cavities of the cup or goggle. An outer rigid surface of the goggle or goggles can seal against a patient's skin around a perimeter of his/her eye sockets using a belt, elastic band, or adhere to it using adhesive tape. The goggle cup is rigid apart from a window of flexible material or an outlet to a secondary chamber that has some flexible walls allowing the change in the volume via mechanical means. Alternatively, the cup is held against the peri-orbital skin using variable force so it can be held tighter or looser resulting in smaller functional orbital volume versus larger orbital volume respectively. Subsequently, when the means to alter functional orbital volume is actuated, a pressure differential from atmospheric pressure can be created and maintained inside the one or more goggle primary chambers and over one or both orbits. The primary chamber pressure can be either increased or decreased relative to atmospheric pressure via changing the chamber volume, depending on the desired therapeutic effect (e.g., whether glaucoma is being treated or whether papilledema is being treated). The primary chamber pressure can be modulated in response to the prerecorded measurements of the change in orbital pressure whether synchronized or asynchronized with the prerecorded waveform. Additionally, the modulation of the functional orbital volume can be determined by other measured vital data of the subject such as pulse oximetry, electric activity of the heart obtained via electrocardiogram (EKG), head position, breathing cycle, blinking, and/or eye movement tracking. Furthermore, the modulation can aim at exaggerating or diminishing the pre-treatment orbital pulsatility. Asynchronized modulation of the chamber volume can be utilized to improve the venous return from the orbit and diminish the episcleral venous pressure of the eyeball to aid in aqueous drainage and subsequently lowering inter ocular pressure.

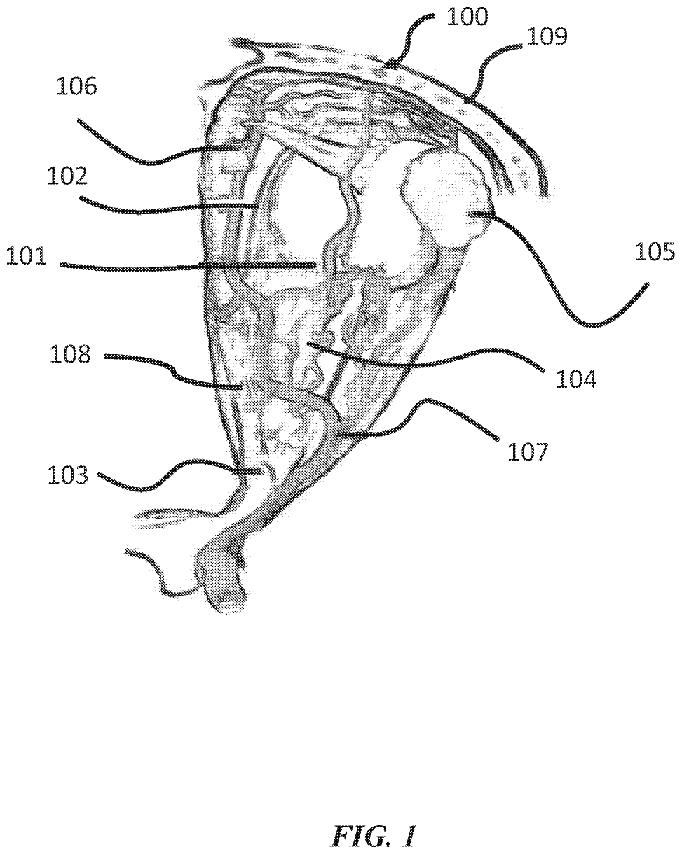

FIG. 1 through FIG. 11 wherein like parts are designated by like reference numerals throughout, illustrate an example embodiment or embodiments of a device and corresponding method of use to address issues with blood flow impairment, abnormal translaminar pressure across the optic nerve head, and poor aqueous humor drainage, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

FIG. 1 illustrates orbital contents in a top cross-section. The orbit 100 contains the eyeball 101 and its adnexal structures such as the extraocular muscles, the thickest of which is the medial rectus 102. The optic nerve 103 connects the eyeball 101 to the brain and is covered with an optic nerve sheath 104. The space between the optic nerve 103 and its sheath 104 is filled with CSF in free communication with the intracranial cavity. The lacrimal gland 105 is in the upper-outer corner of the orbit 100. The superior oblique muscle 106 is another extraocular muscle in the orbit 100. Blood flow to the orbit 100 is provided primarily by the ophthalmic artery 107 and its branches and its venous drainage is primarily through the superior ophthalmic vein 108. The orbital contents are surrounded by the rigid orbital bony walls, except for the front outlet of the orbit 100 that is guarded by the lids and is outlined by the orbital rim 109.

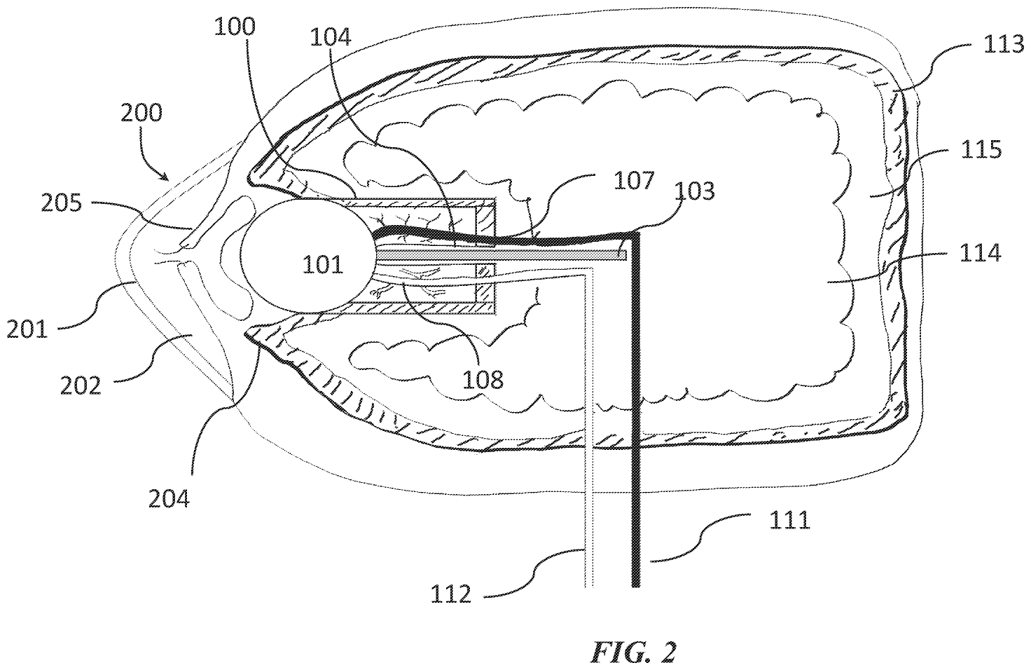

FIG. 2 illustrates a simplified diagram of the orbital contents and their intracranial and extracranial connections. The orbit 100 contains the eyeball 101 and its adnexal structures such as the eyelids 205 and orbital blood vessels. The orbital bony walls are continuous with the bony skull 113. It is also connected to the intracranial cavity through apertures in the bone such as the optic nerve canal, superior orbital fissure, and inferior orbital fissure. The optic nerve 103 connects the eyeball 101 with the brain 114 and is covered by the optic nerve sheath 104 and is surrounded by CSF that is continuous with the intracranial CSF 115. The orbit 100 contains blood vessels such as the ophthalmic artery 107 that is continuous with the internal carotid artery 111 and internal jugular vein 112. With each arterial pulsation following heart contraction, the blood pressure is elevated (systolic phase of blood pressure) that causes the volume of blood within the intracranial contents to increase before it decreases during the diastolic phase (heart relaxation). These heartbeat-to-heartbeat cyclic changes in the blood pressure causes intracranial pulsations of the CSF pressure. Similar pulsations develop in the orbital contents due to the change in the blood volume within the orbit. Furthermore, since a CSF space exists in the orbit underneath the optic nerve sheath, some intracranial CSF pulsatility is transmitted to the orbit. Additionally, the thin-walled veins draining the orbital veins are under the pressure effect of the CSF. The rigid eye cup of the goggle 201 of the device 200 forms a seal around the peri-orbital skin and contains a chamber 202 that is continuous with the orbital contents so that changes in the 202 chamber volume and/or pressure is indicative of the change in the intraorbital content change in volume and or pressure respectively.

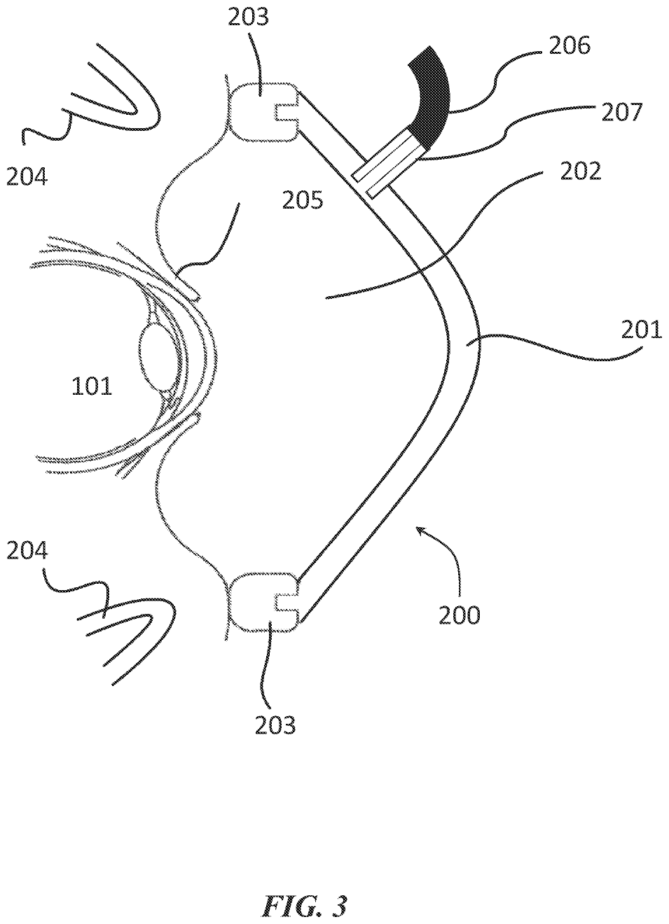

FIG. 3 illustrates a side schematic view of a device 200, in accordance with at least one embodiment, positioned in front of an orbit. The assembly can include an eye cup, eye cups, goggle or goggles 201, shown in cross-section, including one or more primary chambers 202. The body of the goggle or goggles 201 can be relatively rigid, so that it can maintain the differential pressure inside the primary chamber(s) 202, and made of a substantially transparent material, allowing a patient wearing the goggle or goggles 201 to view the external environment such as fixation targets while also allowing the eyeball 101 be viewed by an examiner. There can be a seal material 203 positioned around a perimeter of the goggle or cup body, which can create a seal between edges of the goggle or goggles 201 and the patient's skin around the orbits against the rigid orbital rim 204. The seal material 203 can be a softer rubber or adhesive-coated silicone rubber in close contact with the skin by a strap around the head (not shown) or by its adhesive property.

The primary chamber(s) 202 inside the goggle or goggles 201 is in fluid communication through a port 207 via tubing 206 with a secondary chamber 301 inside a control unit 300 of the device. As used here, “fluid communication”, refers to the unrestricted flow or movement of the fluid (either gas, liquid, or a combination of different gases and liquids) between two or more chambers or compartments. This can be achieved through openings, valves, tubes, or other conduits that allow the fluid to move freely between the chambers. The air pressure inside the secondary chamber 301 is continuously monitored and can have its pressure altered and/or the volume of the chamber altered by the control unit 300 of the device 200. The eyelid 205 is shown over the front of the eye. It can be seen in the schematic illustration of FIG. 3 that the pressure inside the primary chamber(s) 202 is reflective of the pressure inside the orbit transmitted through the surface of the eyeball 101. Alternation of the pressure in the primary chamber(s) 202 and/or alternating its volume can act on the entire area in front of the eye and get transmitted to the contents of the orbit. This altered pressure and/or volume can constantly increase, decreased, modulated and/or cycled to produce therapeutic effect on the orbit and its contents.

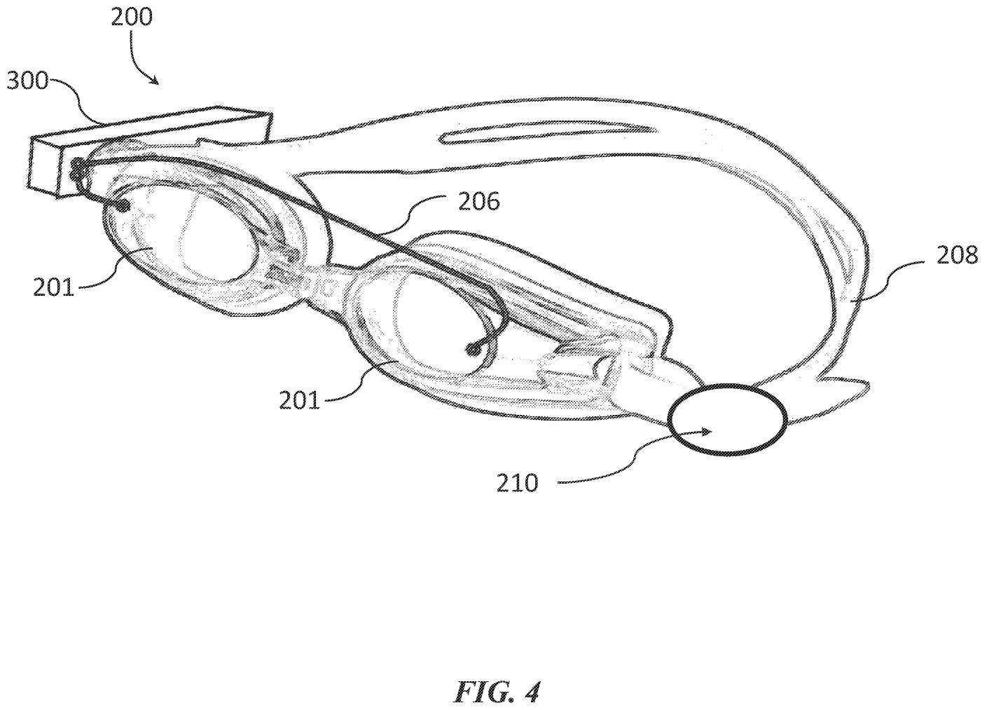

FIG. 4 illustrates a front view of a device 200 in which a volume control unit 300 is mounted to a pair of goggles 201. The control unit 300 incorporates the pressure measuring transducers, pressure and or volume control mechanism, power supply, controlling unit and communication modules. The device 200 can be mounted to the patient head using an adjustable elastic strap 208 with means to dynamically tighten or loosen the strap on each side using the actuator 210. When the actuator 210 controlled with the control unit 300 shortens, the eye cup of the goggles 201 is pushed firmer against the periorbital skin allowing the effective chamber volume to decrease, elevating the pressure behind the primary chamber(s) 202. The chamber of each eye communicates to the control unit 300 via a tube 206.

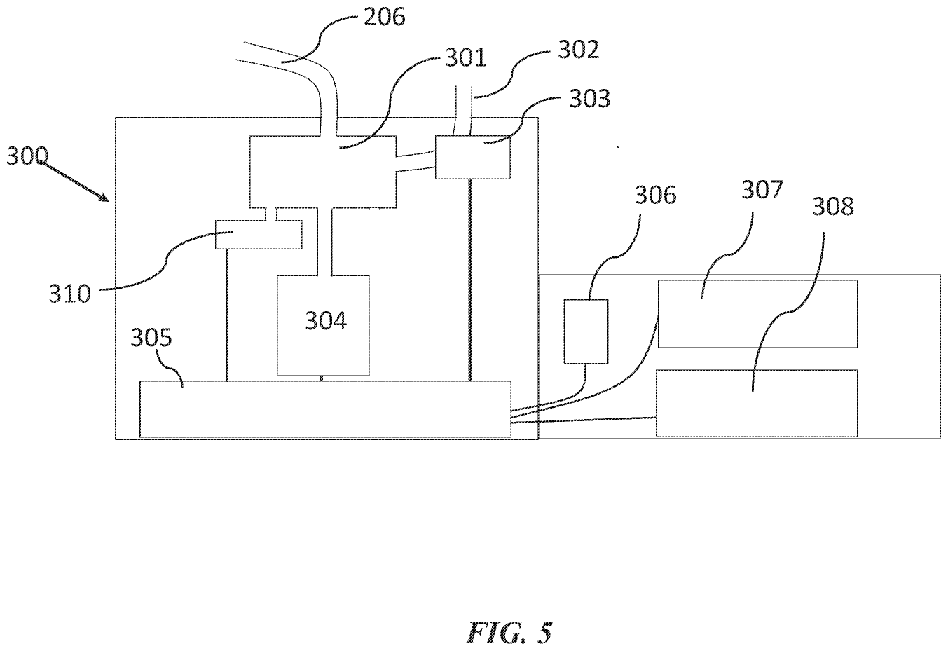

FIG. 5 illustrates a view of the control unit 300 in which a pressure/volume control mechanism is mounted. The control unit 300 contains a secondary chamber 301 for each goggle eye cup primary chamber 202. The secondary chamber 301 is freely communicating with the primary chamber 202 via tube 206 so that the air pressure inside the secondary chamber 301 is equal to the air pressure in the primary chamber 202. Chamber 301 is connected to the sensing outlets of the pressure transducer 310 which communicates with the processing unit 305. The processing unit 305 controls a volume displacement pump 304 which can increase or decrease its volume to modulate the total effective volume of the system made of primary chamber(s) 202, secondary chamber(s) 301 and pump 304. The change in volume results in a change in the pressure of secondary chamber(s) 301 and primary chamber(s) 202 respectively due to the continuity of the system. The processing unit 305 also controls a release valve 303 that is connected in one end to the secondary chamber(s) 301 and the other end is connected with the external atmospheric air via orifice 302. When needed, the control unit can open the valve 303 to reset the pressure in the system to zero or atmospheric pressure. When needed, the valve 303 can be closed to maintain a closed system. The processing unit 305 is connected to a power supply 307, communication module 308 and means for biosensing 306. In an alternative embodiment, the pressure transducer 310, the release valve 303 and the volume displacement pump 304 are all connected directly to the primary chamber(s) 202 omitting the secondary chamber(s) 301.

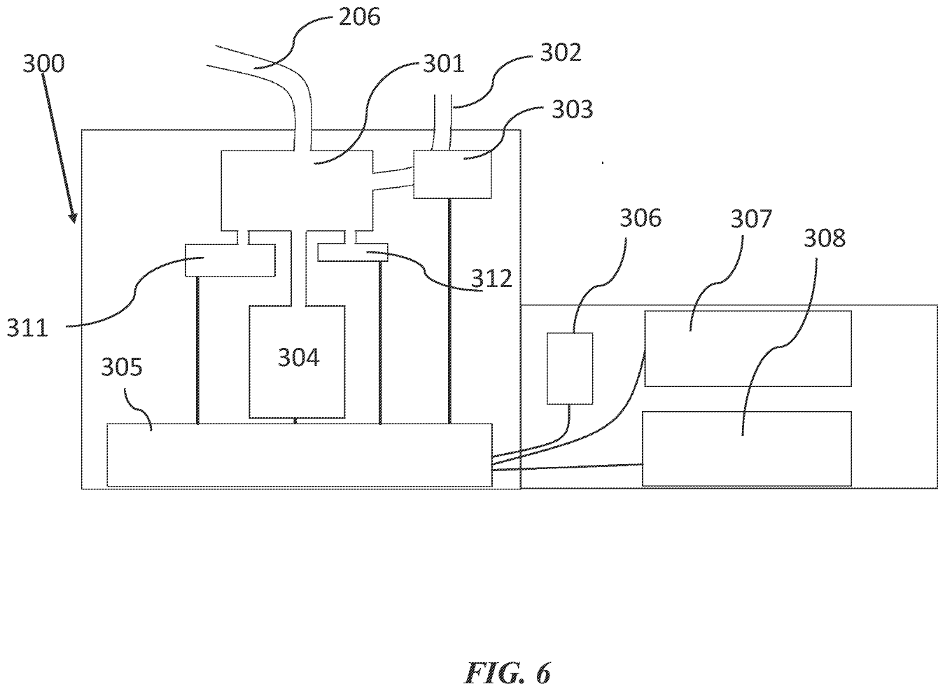

FIG. 6 illustrates a view of an alternative embodiment of the control unit 300 in which a pressure/volume control mechanism is mounted. The control unit 300 contains a secondary chamber 301 per each goggle eye cup chamber 202. The secondary chamber 301 is fluidly communicating with the primary chamber 202 via tube 206 so that the air pressure inside 301 is equal to the air pressure in primary chamber 202. Secondary chamber 301 is connected to the sensing outlets of two transducers, the primary pressure transducer 311 and a secondary pressure transducer 312. The primary sensor provides a wider range of pressure measurements while the secondary transducer has a high sensitivity, and smaller range measurements. Small changes of the orbital volume such as these induced by the change in the volume of blood in the orbit during cardiac cycle cause measurable changes through the higher sensitivity secondary pressure transducer 312 but are too small to trigger the primary transducer 311. Larger volume changes of the orbital contents such as those induced by extraocular muscle movements or eyelid opening or closing will completely saturate the secondary pressure transducer but will still be in range and so measurable by the primary, larger range primary transducer. Both transducers 311, 312 communicate with the processing unit 305. The processing unit 305 controls a volume displacement pump 304 which can increase or decrease its volume to modulate the total effective volume of the system made of chamber 202, secondary chamber 301 and pump 304. The change in volume results in a change in the pressure of the secondary chamber 301 and the primary chamber 202, respectively, due to the continuity of the system. The processing unit 305 also controls a release valve 303 that is connected in one end to the secondary chamber 301 and the other end is connected with the external atmospheric air via orifice 302. When needed, the control unit 300 can open the valve 303 to reset the pressure in the system to zero or atmospheric pressure. When needed, the valve 303 can be closed to maintain a closed system. The processing unit 305 is connected to a power supply 307, communication module 308 and means for biosensing patient vitals parameters 306. The processing unit 305 processes the input values from the pressure transducers 311, 312, biosensing module 306 and controls the volume displacement pump 304, to control and or module the pressure in chamber 301 as needed and preset by the user and display the settings and readings on a built-in monitor and/or send it to an external device such as a computer via the communication module 308. In an alternative embodiment, the primary pressure transducer 311, the secondary pressure transducer 312, the release valve 303 and the volume displacement pump 304 are all connected directly to the primary chamber(s) 202 omitting the secondary chamber 301.

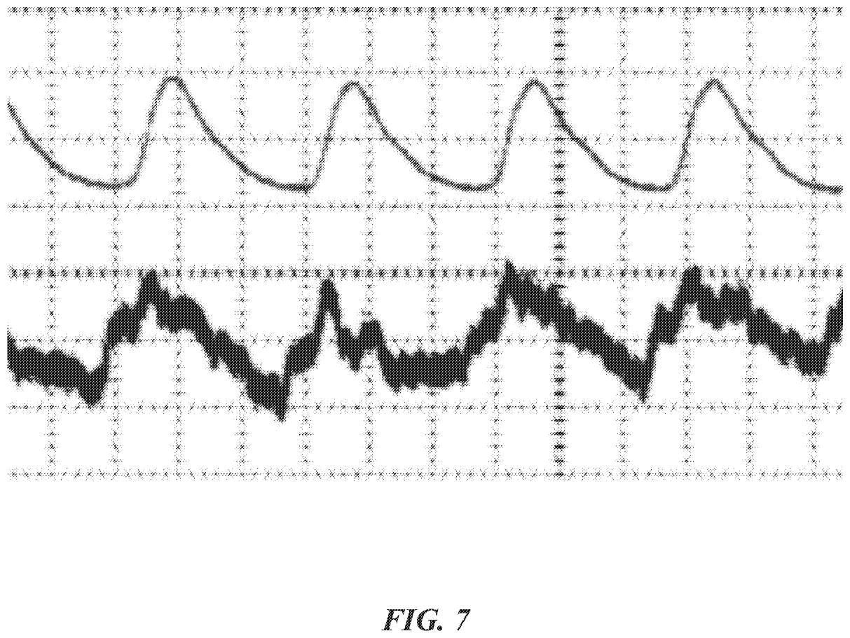

FIG. 7 illustrates the simultaneous recording of a normal subject's pulse oximetry at the fingertip alongside the pressure changes measured by the device 200. The top tracing (thin line graph) demonstrates the change in peripheral blood oxygen saturation which follows the cardiac cycle. The bottom tracing with the thick line is the change of the goggle pressure reading measured in the primary chamber(s) 202 showing similar pulsatile nature indicative of the change in orbital volume along with the cardiac cycle.

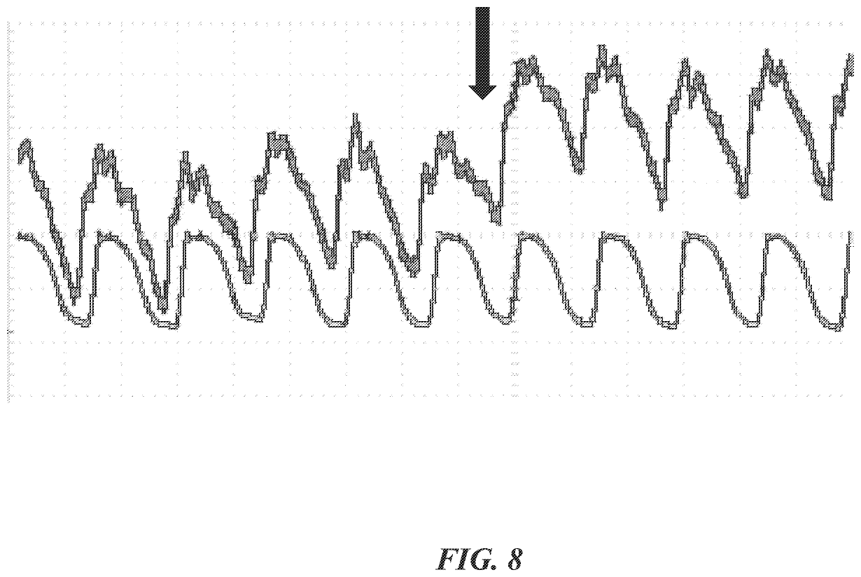

FIG. 8 illustrates the simultaneous recording of a normal subject's pulse oximetry at the fingertip alongside the pressure changes measured by the device 200 initially without jugular vein occlusion followed by jugular vein occlusion. The bottom tracing (thin line graph) demonstrates the change in peripheral blood oxygen saturation which follows the cardiac cycle. The top tracing with the thick line is the change of the goggle pressure reading measured in the primary chamber(s) 202 showing similar pulsatile nature indicative of the change in orbital volume along with the cardiac cycle. To the left of the solid black arrow, the tracing is obtained prior to jugular vein occlusion, once jugular vein occlusion has been applied (indicated by the black arrow), the pressure measured in the primary chamber(s) 202 significantly elevates indicating venous volume expansion in the orbit.

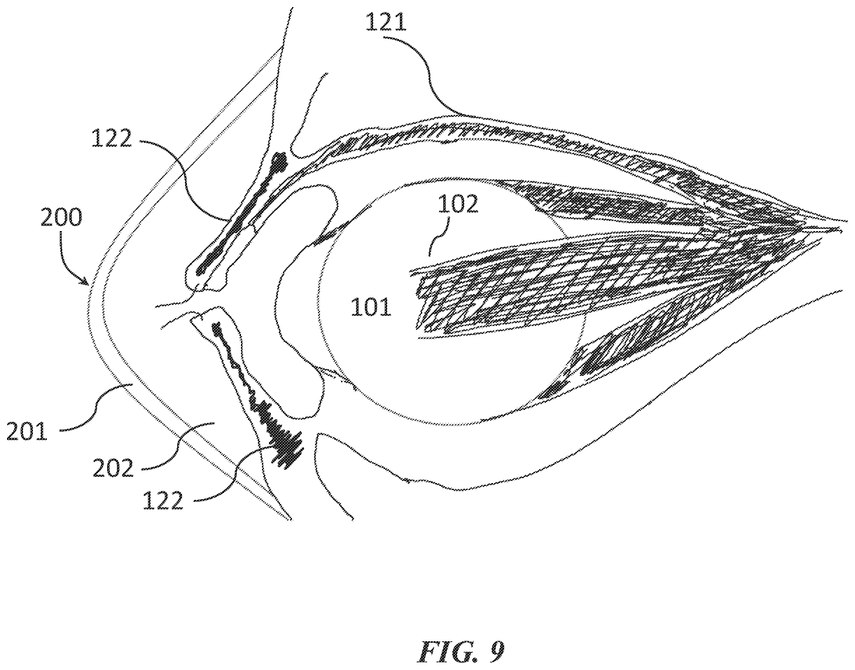

FIG. 9 illustrates a side view of the contents of the orbit 100 with the device 200 placed in front of the orbit 100. The orbit 100 contains the eyeball 101 to which the extraocular muscles are attached. The medial rectus muscle is the thickest one of the 4 straight muscles attached to the eyeball. Its contraction causes the eyeball to turn inwards. The levator muscle 121 is attached at one end to the bones of the orbit and on the other end to the upper eyelid. Its contraction causes the eyelids to open. The opposing muscle is the circular orbicularis oculi muscle 122 that contracts to close the eyelids. When the subject is wearing the device 200 and is instructed to look inwards at an external fixation target, a fixation target mounted to the goggles 201 or just voluntarily converge the eyes, the primary pressure transducer is to read a negative deflection compared to the pressure reading looking forwards caused by the reduction of the volume of the medial rectus muscle on its contraction. Similarly, when the subject is asked to look up, fixate on a fixation target upwards, the primary pressure transducer is to read a negative deflection compared to the pressure reading looking forwards caused by the reduction of the volume of the levator muscle 121 on its contraction. When the subject is asked to close the eye, during spontaneous or reflex blinking, or via electrically stimulating the orbicularis muscle 122 is to read a relatively large negative deflection compared to the baseline pressure reading while the eyelid is open due to the reduction of the volume of the levator muscle 121 on its contraction.

The amplitude of these extraocular muscle contraction induced negative pressure readings can be compared to a normative database or compared to the contralateral side readings to diagnose disorders affecting these muscles. It can also be used to follow up on these conditions. For example, a slow decline in the negative pressure readings following repetitive eyelid opening and closure, electric stimulation of the orbicularis muscle 122, or prolonged upgaze, can be used to diagnose or monitor a condition, such as myasthenia gravis.

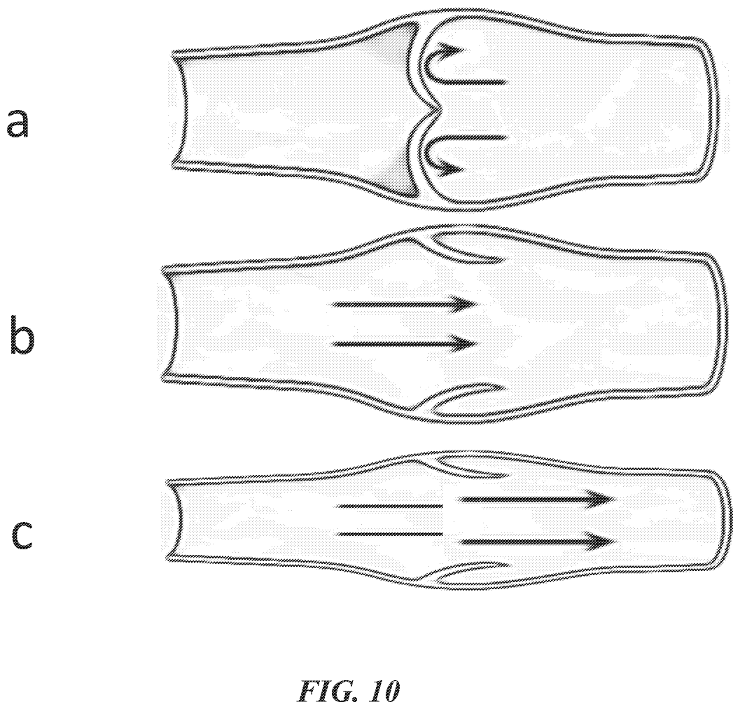

FIG. 10 illustrates cross sections of the superior ophthalmic vein and/or one of its tributaries. In cross section A, the vein is under atmospheric or subatmospheric pressure during the diastolic phase of the heart cycle, the valves within the vein prevent the reflux of blood back towards the contents of the orbit. In cross section B, the vein is under atmospheric or sub atmospheric pressure during the systolic phase of the heart cycle, the arterial pulse pushes the blood through the one way valve(s) and allows the blood to drain from the contents of the orbit to the cavernous venous sinus and eventually to the heart. Cross Section C depicts when an external pressure is applied to the vein walls, it causes the vein to be compressed and push the blood further and faster to the cavernous venous sinus and eventually to the heart. When the external pressure is no longer exerted on the vein wall, the vein returns to its resting status, as shown in cross section A, while the valves prevent the return of the blood to the tissues of the orbit.

The device 200 applies this external pressure to modulate the orbital pressure synchronized or asynchronized with the subject's vitals such as the heart cycle, EKG tracing or pre-measured orbital pulsatility to promote drainage of blood from the orbital contents into the cavernous sinus to promote circulation to the orbit. This method can also be utilized to evacuate the episcleral veins, lower the episcleral venous pressure to lower the intraocular pressure per the Goldmann equation:

$$IOP in millimeters of mercury ( mmHg ) = ( rate of aqueous formation / facility of outflow ) + episcleral venous pressure .$$

The alternation of the external pressure applied by the device 200 to the veins of the orbit and the surface of the eyeball along with the one-way valves of these veins and lymphatic vessels draining the aqueous humour of the eye produces a peripheral heart that sucks the aqueous from the collector aqueous channels into the orbital veins and in turn to the cavernous venous sinus and then the internal jugular vein and the heart.

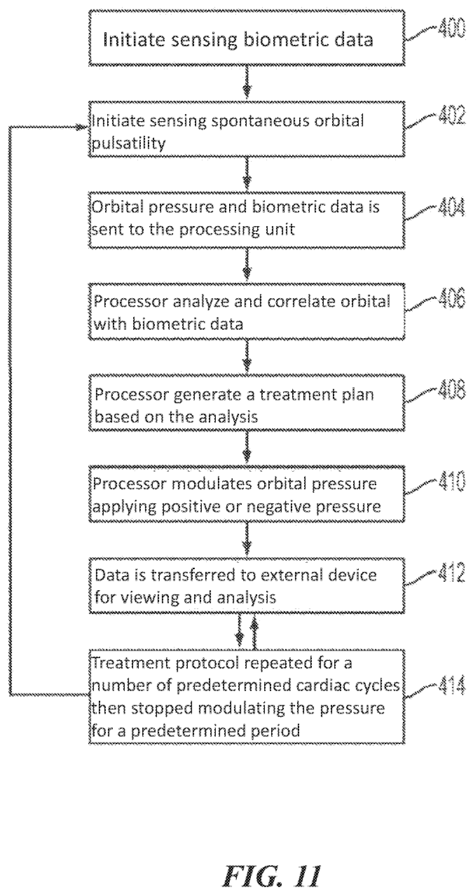

FIG. 11 is a flow chart showing the steps of measuring orbital pulsatility and applying treatment protocol for intended therapeutic effects. The device 200 once triggered by the operator initiates sensing biometric data via a biometric sensor such as pulse oximeter or EKG (step 400). The device 200 then obtains the data from the pressure/volume sensors as previously described (Step 402) and then sends both sets of data biometric and orbital pressure related to the processing unit 305 (step 404). A processor of the processing unit 305 correlated the data and analyzed the data points obtained to detect abnormalities in the orbital pulsatility such as attenuated arterial waveform, delayed arterial filling of the orbital contents, or slow filling (Step 406). Based on the analysis, and the predetermined therapeutic effect selected by the operator, the processor generates a treatment protocol (Step 408). The treatment protocol is applied via having the processing unit 305 control pressure control means to apply constant positive, constant negative or modulated positive alternating with negative pressure to modulate the orbital pressure to augment or attenuate the spontaneous orbital pulsatility waves either synchronized with the wave or asynchronized with the waves to obtain the pre-determined therapeutic effects (step 410). The data obtained by the processing unit 305 as well as real-time measurements of the modulated orbital pulsatility are transferred to an external device such as a computer to view and further analyze the data (step 412). Once the predetermined therapeutic period ends, the device stops modulating the pressure (step 412) and resets the orbital pressure to restart measuring the spontaneous orbital pulsatility (Step 402) and multiple cycles can be repeated depending on the therapeutic indication.

To further describe the present device and methods, a non-limiting list of examples is provided here:

In Example 1, the device 200 can comprise a goggle 201, at least one pressure transducer 310 and a processing unit 305. The goggle 201 can include at least one primary chamber 202 and can be configured to form a seal around the periorbital skin. The pressure transducer 310 can be in fluid communication with the at least primary chamber 202. The processing unit 305 can be connected to an internal monitor or external device to display the pressure changes in the primary chamber(s) 202 contained by the goggles 201 which indirectly indicate the change in the orbital content volume.

In Example 2, the device 200 of Example 1 can optionally be configured such that the device 200 can include a set of goggles 201 including at least one primary chamber 202 and a secondary chamber 301, the secondary chamber 301 being isolated from the at least one primary chamber 202.

In Example 3, the device 200 of Example 2 can optionally be configured such that the pressure transducer 310 is in fluid communication with the at least one primary chamber 202 and the secondary chamber 301.

In Example 4, the device 200 of any one or any combination of Examples 1-3 can optionally be configured such that the pressure transducer 310 includes 2 transducers 311, 312 one for low pressure and one for high pressure measurements.

In Example 5, the device 200 of any one or any combination of Examples 1˜4 can optionally further comprise at least one transducer 310 configured to measure a pressure in the at least one primary chamber 202 and electronically communicate the pressure to the processing unit 305.

In Example 6, the device 200 of Example 5 can optionally be configured such that the at least one transducer 310 is a pressure sensor configured to monitor the change in orbital pressure.

In Example 7, the device 200 of any one or any combination of Examples 1-6 can optionally be configured such that the processing unit 305 comprises a control circuit configured to display or transmit at least one primary chamber-based readings to an external device such as a computer.

In Example 8, the device 200 of any one or any combination of Examples 1-7 can optionally further comprise a battery power supply 307 configured to provide power to operate a pump 304 and the processing unit 305.

In Example 9, the device 200 of any one or any combination of Examples 1-8 can optionally be configured such that the goggle 201 includes one or more vents 303 fluidly coupling the at least one primary chamber 202 and a surrounding environment.

In Example 10, the device 200 of any one or any combination of Examples 1-9 can optionally further comprise a seal material 203 coupled to a perimeter of the goggle 201.

In Example 11, a method can comprise placing a device 200, including a goggle 201 having at least one primary chamber 202, a volume displacement pump 304 in fluid communication with the at least one primary chamber 202, and a control unit 300, over and around an eye, including spacing the goggle from a surface of the eye; setting a static target pressure within the at least one primary chamber using the control mechanism; establishing the target pressure within the at least one primary chamber; and maintaining the target pressure within the at least one primary chamber for a period of time.

In Example 12, the method of Example 11 can optionally further comprise adjusting the target pressure.

In Example 13, the method of any one or any combination of Examples 11 or 12 can optionally further comprise modulating the target pressure based, at least in part, upon measurement of at least one physiological parameter to match or antagonize the blood flow.

In Example 14, the method of any one or any combination of Examples 11-13 can optionally be configured such that establishing the target pressure includes creating a pressure differential from atmospheric pressure in the at least one primary chamber.

In Example 15, the method of Example 14 can optionally be configured such that creating the pressure differential from atmospheric pressure in the at least one primary chamber includes changing a volume of some collapsible intraorbital contents such as blood vessels to produce peristalsis and improve blood flow.

In Example 16, the method of any one or any combination of Examples 11-15 can optionally be configured such that maintaining the target pressure includes maintaining a pressure in an operating range from −30 mmHg to 30 mmHg.

In Example 17, the method of Example 16 can optionally be configured such that maintaining the pressure in the operating range includes maintaining the pressure in an operating range from 5 mmHg to 20 mmHg (gage).

In Example 18, the method of any one or any combination of Examples 11-17 can optionally be configured such that maintaining the target pressure includes reducing an orbital pressure of the eye.

In Example 19, the method of any one or any combination of Examples 11-19 can optionally be configured such that maintaining the target pressure includes inhibiting a progression of glaucoma associated with the eye.

To any extent utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may existing in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A device for measuring and modifying orbital content volume, pressure, blood flow, venous drainage, lymphatic drainage, and pulsatility of a subject's eye socket, the device comprising:
a goggle comprising:
at least one primary chamber configured to form a seal around periorbital skin of the subject; and
a strap configured to mount the goggle on a subject's head, the strap having an actuator; and
a control unit in fluid communication with the at least one primary chamber and in electrical communication with the actuator, the control unit configured to:
monitor a pressure inside the at least one primary chamber; and
control the actuator to tighten or loosen the strap to increase or decrease the pressure inside the at least one primary chamber.

2. The device of claim 1, wherein a body of the goggle is generally rigid.

3. The device of claim 2, wherein the body comprises a substantially transparent material.

4. The device of claim 1, further comprising a seal material coupled to a perimeter of the goggle.

5. The device of claim 4, wherein the seal material comprises softer rubber or adhesive-coated silicone rubber.

6. The device of claim 1, wherein the control unit is in fluid communication with a port in the at least one primary chamber via tubing.

7. A device for measuring and modifying orbital content volume, pressure, blood flow, venous drainage, lymphatic drainage, and pulsatility of a subject's eye socket, the device comprising:
a goggle comprising at least one primary chamber configured to form a seal around periorbital skin of the subject; and
a control unit comprising:
at least one pressure transducer in fluid communication with the at least one primary chamber configured to monitor pressure in the at least one primary chamber;
a pump in fluid communication with the at least one primary chamber; and
a processing unit in electrical communication the at least one pressure transducer and pump, the processing unit configured to control the pump to increase or decrease a pressure in the at least one primary chamber based on communication from the least one pressure transducer.

8. The device of claim 7, wherein the control unit further comprises a release valve in fluid communication with the at least one primary chamber and in electrical communication with the processing unit, and wherein the processing unit is further configured to control the release valve.

9. The device of claim 7, wherein the control unit further comprises a secondary chamber in fluid communication with the at least one primary chamber, and wherein the at least one pressure transducer and fluid pump are in fluid communication with the secondary chamber.

10. The device of claim 7, wherein the device further comprises one or more of: a power supply, communication module, and means for biosensing.

11. The device of claim 7, wherein the at least one pressure transducer comprises two transducers wherein one transducer is for low pressure measurements and one transducer is for high pressure measurements.

12. A method for measuring pressure changes induced by voluntary or involuntary eye movement, the method comprising:
providing a device comprising:
a goggle comprising at least one primary chamber configured to form a seal around periorbital skin of a subject; and
a control unit comprising:
at least one pressure/volume sensor in fluid communication with the at least one primary chamber configured to monitor pressure in the at least one primary chamber;
a pressure control means in fluid communication with the at least one primary chamber; and
a processing unit in electrical communication with the at least one pressure/volume sensor and pressure control means, the processing unit configured to control a pump to increase or decrease a pressure in the at least one primary chamber based on communication from the at least one pressure/volume sensor;
placing the device over an eye of the subject;
initiating sensing biometric data via a biometric sensor;
having the subject move their eyes;
obtaining data from the pressure/volume sensors;
sending data to the processing unit; and
correlating and analyzing the data, by the processing unit, to detect a change in pressure correlating to an eye movement.

13. The method of claim 12, wherein the eye movement is voluntary or involuntary.

14. The method of claim 12, wherein having the subject move their eyes comprises having the subject look at a fixation target.

15. The method of claim 12, wherein the movement of the subject's eyes comprises the subject converging their eyes and the change in pressures is a reduction in pressure corresponding to a reduction of a volume of extraocular rectus muscles during contraction.

16. The method of claim 12, wherein the movement of the subject's eyes comprises the subject looking up and the change in pressures is a reduction in pressure corresponding to a reduction of a volume of a levator muscle during contraction.

17. The method of claim 12, wherein the movement of the subject's eyes comprises the subject closing their eye and the change in pressures is a reduction in pressure corresponding to a reduction of a volume of a orbicularis oculi muscle contraction.

18. A method for measuring orbital pulsatility and applying treatment protocol for predetermined therapeutic effects, the method comprising:
providing a device comprising:
a goggle comprising at least one primary chamber configured to form a seal around periorbital skin of a subject; and
a control unit comprising:
at least one pressure/volume sensor in fluid communication with the at least one primary chamber configured to monitor pressure in the at least one primary chamber;
a pressure control means in fluid communication with the at least one primary chamber; and
a processing unit in electrical communication with the at least one pressure/volume sensor and pressure control means, the processing unit configured to control a pump to increase or decrease a pressure in the at least one primary chamber based on communication from the at least one pressure/volume sensor;
placing the device over an eye of the subject;
initiating sensing biometric data via a biometric sensor;
obtaining data from the pressure/volume sensors;
sending both sets of data to the processing unit;
correlating and analyzing the data, by the processing unit, to detect abnormalities in the orbital pulsatility;
generating, by the processing unit, a treatment protocol based on analysis and a predetermined therapeutic effect;
applying the treatment protocol via having the processing unit control pressure control means to apply constant positive, constant negative or modulated positive alternating with negative pressure to modulate the orbital pressure to augment or attenuate spontaneous orbital pulsatility waves either synchronized with the wave or asynchronized with the waves to obtain the predetermined therapeutic effect; and
transferring the data obtained by the processing unit as well as real-time measurements of the modulated orbital pulsatility to an external device.

19. A device for measuring and modifying orbital content volume, pressure, blood flow, venous drainage, lymphatic drainage, and pulsatility of a subject's eye socket, the device comprising:
a goggle comprising at least one primary chamber configured to form a seal around periorbital skin of the subject;
at least one pressure transducer in fluid communication with the at least one primary chamber configured to measure a pressure in the at least one primary chamber and electronically communicate the pressure to a processing unit; and
the processing unit connected to an internal monitor or external device to display pressure changes in the primary chamber contained by the goggle, which indirectly indicates a change in orbital content volume.

20. The device of claim 19, further comprising a secondary chamber, the secondary chamber being isolated from the at least one primary chamber.

21. The device of claim 20, wherein the pressure transducer is in fluid communication with the at least one primary chamber and the secondary chamber.

22. The device of claim 19, wherein the pressure transducer comprises two transducers, and wherein one transducer is for low pressure measurements and one transducer is for high pressure measurements.

23. The device of claim 19, wherein the at least one transducer is a pressure sensor configured to monitor change in orbital pressure.

24. The device of claim 19, wherein the processing unit comprises a control circuit configured to display or transmit at least one primary chamber-based reading to an external device.

25. The device of claim 19, further comprising a battery power supply configured to provide power to operate a pump and a control unit.

26. The device of claim 19, wherein the goggle comprises one or more vents fluidly coupling the at least one primary chamber and a surrounding environment.

27. The device of claim 19, further comprising a seal material coupled to a perimeter of the goggle.

28. A method for modifying orbital content volume, pressure, blood flow, venous drainage, lymphatic drainage, and pulsatility of a subject's eye socket, the method comprising:
placing a device comprising a goggle having at least one primary chamber, a volume displacement pump in fluid communication with the at least one primary chamber, and a control unit, over and around an eye, including spacing the goggle from a surface of the eye;
setting a static target pressure within the at least one primary chamber using a control mechanism;
establishing the target pressure within the at least one primary chamber; and
maintaining the target pressure within the at least one primary for a period of time.

29. The method of claim 28, further comprising adjusting the target pressure.

30. The method of claim 28, further comprising modulating the target pressure based, at least in part, upon measurement of at least one physiological parameter to match or antagonize blood flow.

31. The method of claim 28, wherein establishing the target pressure comprises creating a pressure differential from atmospheric pressure in the at least one primary chamber.

32. The method of claim 31, wherein creating the pressure differential from atmospheric pressure in the at least one primary chamber comprises changing a volume of some collapsible intraorbital contents to produce peristalsis and improve blood flow.

33. The method of claim 28, wherein maintaining the target pressure comprises maintaining a pressure in an operating range from −30 mmHg to 30 mmHg.

34. The method of claim 33, wherein maintaining the pressure in the operating range includes maintaining the pressure in an operating range from 5 mmHg to 20 mmHg.

35. The method of claim 28, wherein maintaining the target pressure includes reducing an orbital pressure of the eye.

36. The method of claim 28, wherein maintaining the target pressure includes inhibiting a progression of glaucoma associated with the eye.
